(19) United States
(12) Patent Application Publication
(10) Pub. No.: US 2025/0051578 A1
(43) Pub. Date: Feb. 13, 2025

(54) RHODAMINE-METHYLENE BLUE DERIVATIVE FLUORESCENT PROBE AND PREPARATION METHOD AND APPLICATION THEREOF

(71) Applicant: Henan Polytechnic University, Jiaozuo (CN)

(72) Inventors: Wei-Na Wu, Jiaozuo (CN); Yuan Wang, Jiaozuo (CN)

(21) Appl. No.: 18/599,314

(22) Filed: Mar. 8, 2024

Related U.S. Application Data
(63) Continuation of application No. PCT/CN2023/112569, filed on Aug. 11, 2023.

(51) Int. Cl. C09B 21/00; G01N 21/64; G01N 33/58

(52) U.S. Cl. CPC C09B 21/00; G01N 21/6428; G01N 21/6456; G01N 33/582; G01N 2021/6439

(57) ABSTRACT

A rhodamine-methylene blue derivative fluorescent probe and its preparation method and application are in the technical field of organic synthesis. The rhodamine-methylene blue derivative fluorescent probe has available raw materials and a simple prepared method; the rhodamine-methylene blue derivative fluorescent probe can detect ClO⁻ and ATP in cells by synchronized dual-channel fluorescence, with a detection limit of 0.90 nM for the ClO⁻ and 23.60 nM for the ATP, and thus the rhodamine-methylene blue derivative fluorescent probe can be used for fluorescence quantitative detection of the ClO⁻ and the ATP, and can also be used for simultaneous fluorescence imaging of ClO⁻ and ATP in drug-stimulated cells. Therefore, the rhodamine-methylene blue derivative fluorescent probe has a wide range of potential applications.

### TECHNICAL FIELD

The disclosure relates to the technical field of organic synthesis, and more particularly to a rhodamine-methylene blue (C₅₁H₆₀N₈O₃S) derivative fluorescent probe and its preparation method and application.

### BACKGROUND

Hypochlorous acid (HClO)/hypochlorite (ClO⁻) is widely used as a disinfectant in daily life. At the same time, the ClO⁻ is also an important reactive oxygen species (ROS) produced during aerobic cellular respiration and participates in numerous physiological processes. Cells synthesize adenosine triphosphate (ATP) through oxidative phosphorylation to provide energy for cellular activities during the aerobic cellular respiration, and a level of the ATP is also a sensitive signal of cellular health status. The ClO⁻ and the ATP in cells have a highly close interconnection, and dynamic balance and fluctuation regulation of the ClO⁻ and the ATP play a crucial role in life processes such as survival, growth, aging and death of creatures. Dysfunctions such as abnormal oxidative stress and disturbed energy metabolism in cells are important causative factors for formation and metastasis of lesions in a variety of malignant diseases (e.g., Alzheimer's disease, cardiovascular disease, epilepsy, and cancer). Therefore, development of effective tools to monitor cellular ClO⁻ and ATP levels is particularly important to help elucidate their physiological relationships and related disease pathogenesis.

In recent years, fluorescent molecular probe technology has become a mainstream means of biological detection due to its high sensitivity, simple operation and low cost. However, most of current fluorescent probes can only detect the ClO⁻ or the ATP alone, and cannot detect both the ClO⁻ and the ATP at the same time, limiting their further practical applications.

### SUMMARY

The disclosure provides a rhodamine-methylene blue derivative fluorescent probe and its preparation method and application, to solve problems in related art.

To achieve above purposes, the disclosure provides technical solutions as follows.

The disclosure provides a rhodamine-methylene blue derivative fluorescent probe, having the structural formula as below:

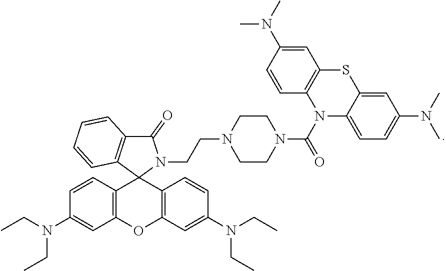 [SMILES: CCN(CC)c1ccc2c(c1)Oc1cc(N(CC)CC)ccc1C21c2ccccc2C(=O)N1CCN1CCN(C(=O)N2c3ccc(N(C)C)cc3Sc3cc(N(C)C)ccc32)CC1]

The disclosure further provides a preparation method of the rhodamine-methylene blue derivative fluorescent probe, includes: methylene blue-carbonyl chloride (C₁₇H₁₈ClN₃OS, MB-COCl) and sodium carbonate (Na₂CO₃) are dissolved in an organic solvent to obtain a first solution, an organic solution including rhodamine B-(2-aminoethyl) piperazine (C₃₄H₄₃N₅O₂, RhB-AP) is added to the first solution to obtain a second solution, the second solution is stirred for reaction to obtain a reacted solution, the reacted solution is added to iced water and then extracted to obtain an extracted product, organic phases are separated from the extracted product followed by removing a solvent by rotary evaporation from the organic phases, and then the organic phases are purified to obtain the rhodamine-methylene blue derivative fluorescent probe.

In an embodiment, a molar ratio of the MB-COCl to the Na₂CO₃ is 1:3.

In an embodiment, a weight-volume ratio of the MB-COCl to the organic solvent is 1 gram (g): 10 milliliters (mL), and the organic solvent includes dichloromethane (CH₂Cl₂).

In an embodiment, a molar ratio of the RhB-AP to the MB-COCl is 4:1, and a solvent for the organic solution including the RhB-AP includes dichloromethane at a concentration of 2.296 mole per liter (mol·L⁻¹).

In an embodiment, conditions of the stirring process are stirring the second solution for 5 hours (h) at room temperature with a stirring frequency of 600 revolutions per minute (rpm).

In an embodiment, ethyl acetate (C₄H₈O₂) is used for extracting the reacted product.

In an embodiment, conditions for the rotary evaporation includes: a temperature of 50 degrees Celsius (° C.), and a pressure of 0.1 megapascal (MPa), and silica gel column chromatography is used for purifying the organic phases.

In an embodiment, an eluent for the silica gel column chromatography includes the ethyl acetate and petroleum ether with a volume ratio (i.e., volume per volume (v:v)) of 1:300.

The disclosure further provides an application of the rhodamine-methylene blue derivative fluorescent probe in detecting ClO⁻ and ATP, such as detecting optical properties of the ClO⁻ and the ATP.

The disclosure further provides an application of the rhodamine-methylene blue derivative fluorescent probe as a ClO⁻ and ATP fluorescent probe in cell imaging, such as detecting the ClO⁻ and the ATP in cells.

The disclosure provides the following technical effects:

The discourse prepares the rhodamine-methylene blue derivative fluorescent probe with readily available raw materials and a simple synthesis method; the rhodamine-methylene blue derivative fluorescent probe can detect the ClO⁻ and the ATP in cells by synchronized dual-channel fluorescence, with a detection limit of 0.90 nanomolar (nM) for the ClO⁻ and 23.60 nM for the ATP, and thus the rhodamine-methylene blue derivative fluorescent probe can be used for fluorescence quantitative detection of the ClO⁻ and the ATP, and can also be used for simultaneous fluorescence imaging of the ClO⁻ and the ATP in drug-stimulated cells. Therefore, the rhodamine-methylene blue derivative fluorescent probe has a wide range of potential applications.

### BRIEF DESCRIPTION OF DRAWINGS

To more clearly illustrate technical solutions in embodiments or the related art, accompanying drawings to be used in the embodiments are briefly described below, and it is apparent that the accompanying drawings in following description are only some of the embodiments of the disclosure, and for those skilled in the art, other accompanying drawings can be obtained based on the accompanying drawings without creative labor.

FIG. 1 illustrates a schematic protium nuclear magnetic resonance (¹H NMR) spectrogram of a rhodamine-methylene blue derivative fluorescent probe prepared in an embodiment 1 of the disclosure.

FIG. 2 illustrates a schematic carbon-13 nuclear magnetic resonance (¹³C NMR) spectrogram of the rhodamine-methylene blue derivative fluorescent probe prepared in the embodiment 1 of the disclosure.

FIG. 3 illustrates a schematic mass spectrogram of the rhodamine-methylene blue derivative fluorescent probe prepared in the embodiment 1 of the disclosure.

FIG. 4A illustrates a schematic fluorescence spectrogram of analytes with a concentration of 1×10⁻⁴ mol/L separately added with a solution of ethanol:phosphate-buffered saline (EtOH:PBS) (0.05 mol/L, pH=7.4, 5:95, v:v) of the rhodamine-methylene blue derivative fluorescent probe (1×10⁻⁵ mol/L) prepared in the embodiment 1 of the disclosure; the analytes respectively include fluorine ion (F⁻), chloride ion (Cl⁻), bromide ion (Br⁻), iodide ion (I⁻), perchlorate (ClO₄⁻), phosphate (PO₄³⁻), dihydrogen phosphate (H₂PO₄⁻), hydrogen phosphate (HPO₄²⁻), pyrophosphate (ppi), sulphate (SO₄²⁻), bisulfite (HSO₃⁻), sulfide ion (S²⁻), calcium ion (Ca²⁺), magnesium ion (Mg²⁺), potassium ion (K⁺), L-cysteine (L-Cys), glutathione (GSH), ClO⁻, hydrogen peroxide (H₂O₂), peroxynitrite (ONOO⁻), singlet oxygen (¹O₂), and peroxyl radical (ROO⁻); and an excitation wavelength is 630 nanometers (nm).

FIG. 4B illustrates a schematic fluorescence spectrogram of analytes with a concentration of 1×10⁻⁴ mol/L separately added with a solution of EtOH:PBS (0.02 mol/L, pH=7.4, 5:95, v:v) of the rhodamine-methylene blue derivative fluorescent probe (1×10⁻⁵ mol/L) prepared in the embodiment 1 of the disclosure; the analytes respectively include PO₄³⁻, H₂PO₄⁻, HPO₄²⁻, ppi, L-Cys, GSH, ATP, adenosine diphosphate (ADP), adenosine monophosphate (AMP), guanosine triphosphate (GTP), cytidine triphosphate (CTP) and uridine triphosphate (UDP); and an excitation wavelength is 360 nm.

FIG. 5A illustrates a schematic fluorescence spectrogram of different concentrations of the ClO⁻ titrated by the solution of EtOH:PBS (0.02 mol/L, pH=7.4, 5:95, v:v) of the rhodamine-methylene blue derivative fluorescent probe (1×10⁻⁵ mol/L) prepared in the embodiment 1 of the disclosure, and illustrates a linear variation trend of fluorescence intensity at 680 nm with concentrations of the ClO⁻; and an excitation wavelength is 630 nm.

FIG. 5B illustrates a schematic fluorescence spectrogram of different concentrations of the ATP titrated by the solution of EtOH:PBS (0.02 mol/L, pH=7.4, 5:95, v:v) of the rhodamine-methylene blue derivative fluorescent probe (1×10⁻⁵ mol/L) prepared in the embodiment 1 of the disclosure, and illustrate a linear variation trend of fluorescence intensity at 590 nm with concentrations of the ATP; and an excitation wavelength is 360 nm.

FIG. 6 illustrates a schematic diagram of Raw246.7 cell imaging, in which “a” represents yellow channel fluorescence imaging, “b” represents near-infrared spectroscopy (NIR) channel fluorescence imaging, “c” represents bright field imaging, “d” represents yellow channel and NIR channel (Yellow+NIR) merged imaging, and “e” represents yellow channel, NIR channel and bright field merged imaging.

FIG. 7 illustrates a schematic diagram of Raw246.7 cell imaging, in which “a” represents the yellow channel fluorescence imaging, “b” represents the NIR channel fluorescence imaging, “c” represents the bright field imaging, “d” represents shows the Yellow+NIR merged imaging, and “e” represents the yellow channel, NIR channel and bright field merged imaging.

### DETAILED DESCRIPTION OF EMBODIMENTS

Specific embodiments of the disclosure are described in detail, detailed descriptions below should not be considered as a limitation of the disclosure, but rather should be understood as more detailed descriptions of certain aspects, features and embodiments of the disclosure.

It should be understood that terms described in the disclosure are only intended to describe particular embodiments and are not intended to limit the disclosure. Furthermore, for a range of values in the disclosure, it should be understood that each intermediate value between upper and lower limits of the range is also specifically disclosed. Each smaller range between any stated value or intermediate value within stated range and any other stated value or intermediate value within the stated range is also included within the disclosure. The upper and lower limits of these smaller ranges may be independently included or excluded from the range.

Unless otherwise indicated, all technical and scientific terms used in the disclosure have same meaning as commonly understood by those skilled in the art. While only methods and materials in the embodiments are described, any methods and materials similar or equivalent to those described may also be used in implementation or testing of the disclosure. All literature referred to in this specification is incorporated by reference for a purpose of disclosing and describing methods and/or materials associated with the literature. In an event of a conflict with any incorporated literature, contents of this specification shall prevail.

It is apparent to those skilled in the art that various improvements and variations may be made to specific embodiments of the specification of the disclosure without departing from scope or spirit of the disclosure. Other embodiments obtained from the specification of the disclosure will be apparent to those skilled in the art. The specification and embodiments are exemplary only.

As used in the specification, terms “contain”, “include”, and “have”, etc., are all open-ended terms, i.e., they are meant to include but not be limited to.

Technical solutions described in the disclosure are conventional in the art, if not otherwise specified. And reagents or raw materials in the disclosure, if not otherwise specified, are purchased from commercial sources or are publicly available.

### Embodiment 1

A preparation method of a rhodamine-methylene blue derivative fluorescent probe includes the following steps.

MB-COCl (1 g, 2.87 millimole (mmol)) and Na₂CO₃ (0.91 g, 8.61 mmol) are added with dichloromethane (10 mL) to obtain a first solution, and then a dichloromethane solution (5 mL) including RhB-AP (6.357 g, 11.48 mmol) is added to the first solution to obtain a second solution, the second solution is stirred for reaction at room temperature with a frequency of 600 rpm for 5 h to obtain a reacted solution, the reacted solution is added to iced water (200 mL) and then extracted by ethyl acetate (3×150 mL) to obtain an extracted product, organic phases are separated from the extracted product followed by removing a solvent by rotary evaporation with conditions including a temperature of 50° C. and a pressure of 0.1 MPa from the organic phases and then organic phases is purified by silica gel column chromatography with an eluent (ethyl acetate:petroleum ether=1:300, v:v) to obtain the rhodamine-methylene blue derivative fluorescent probe in 23% yield.

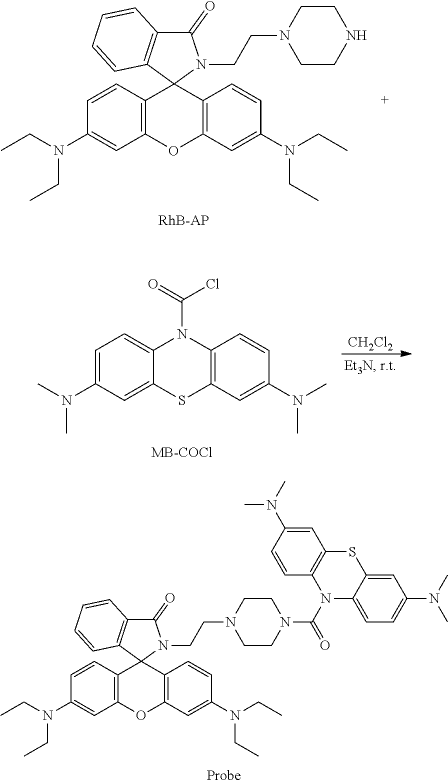

The rhodamine methylene blue derivatives are analyzed by a nuclear magnetic resonance (NMR) instrument and results are as follows:

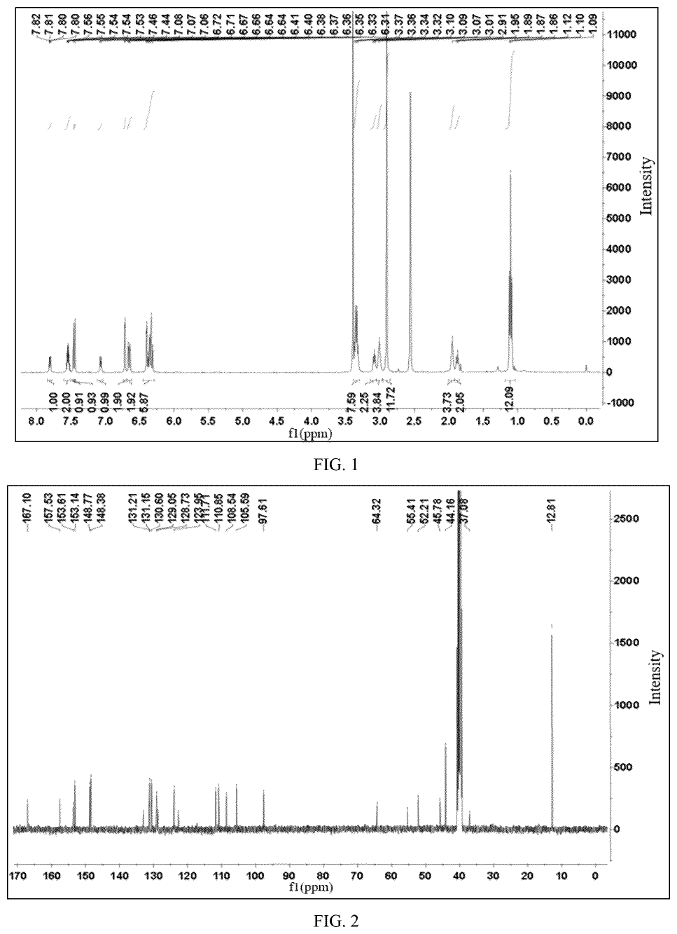

¹H NMR (400 MHz, DMSO-d₆) δ 7.80-7.82 (m, 1H, Ar—H), 7.52-7.57 (m, 2H, Ar—H), 7.46 (s, 1H, Ar—H), 7.44 (s, 1H, Ar—H), 6.71-6.72 (d, 2H, Ar—H), 6.64-6.67 (dd, 2H, Ar—H), 6.31-6.41 (m, 6H, Ar—H), 3.32-3.37 (q, 8H, 4CH₂—CH₃), 3.07-3.10 (t, 2H, CH₂), 3.07-3.10 (t, 2H, CH₂), 3.00-3.03 (t, 4H, 2CH₂), 2.91 (s, 12H, 4CH₃), 1.94-1.97 (t, 4H, 3CH₂), 1.86-1.89 (t, 2H, CH₂), 1.09-1.12 (t, 12H, 4CH₃—CH₂). A specific ¹H NMR spectrogram is shown in FIG. 1.

¹³C NMR (101 MHz, DMSO-d₆) δ 167.10, 157.53, 153.61, 153.14, 148.77, 148.38, 131.21, 131.15, 130.60, 129.05, 128.73, 123.95, 111.71, 110.85, 108.54, 105.59, 97.61, 64.32, 55.41, 52.21, 45.78, 44.16, 37.08, 12.81. A specific ¹³C NMR spectrogram is shown in FIG. 2.

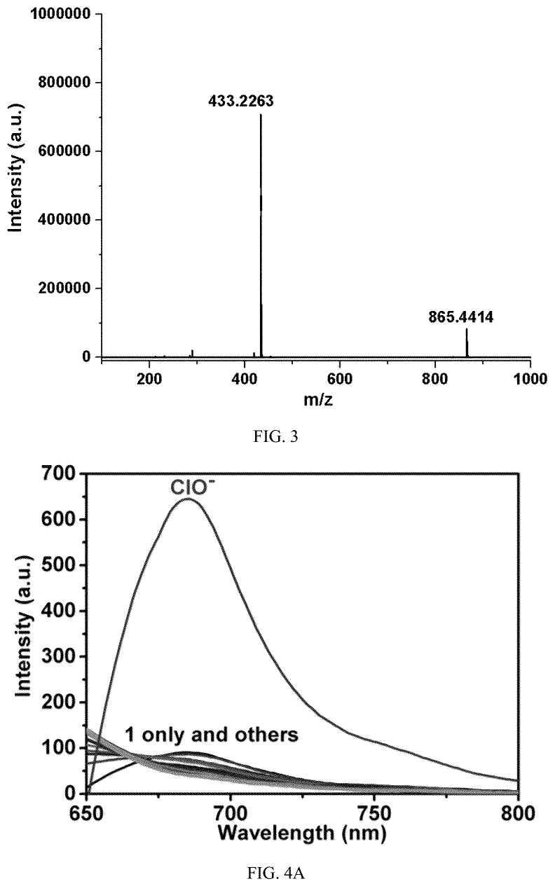

Mass spectra ESI-MS: m/z=865.4414 ([M+H]⁺, calc. 865.4587), 433.2263 for ([M+2H]²⁺, calc. 433.2333). A specific mass spectrogram is shown in FIG. 3.

### Embodiment 2

Optical properties of ClO⁻ and ATP are detected by a rhodamine-methylene blue derivative fluorescent probe.

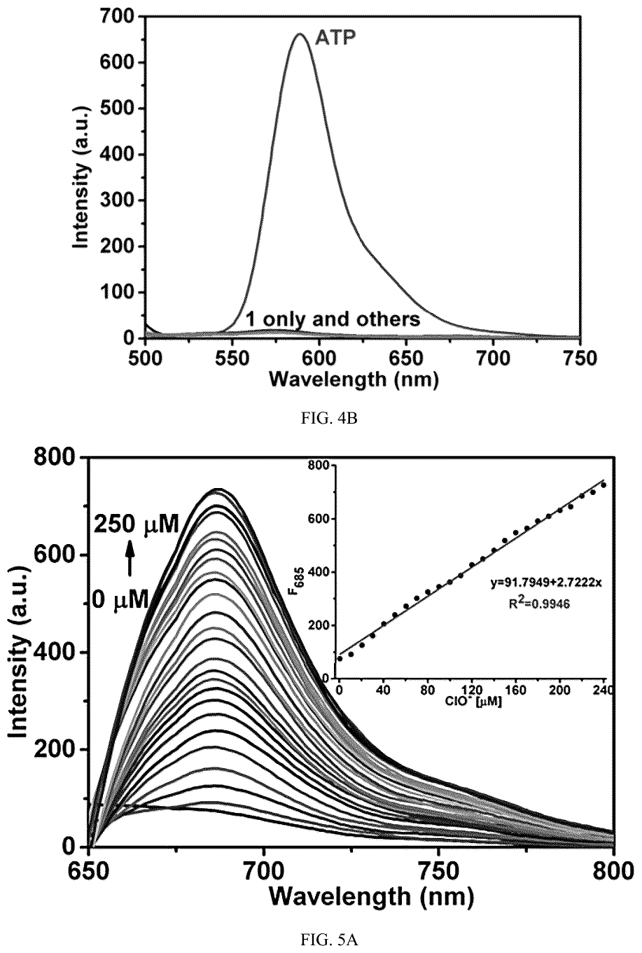

Solutions of EtOH:PBS (0.05 mol/L, pH=7.4, 5:95, v:v) of the rhodamine-methylene blue derivative fluorescent probe (1×10⁻⁵ mol/L) are respectively added with an analyte F⁻ (2.5×10⁻⁴ mol/L), an analyte Cl⁻ (2.5×10⁻⁴ mol/L), an analyte Br⁻ (2.5×10⁻⁴ mol/L), an analyte I⁻ (2.5×10⁻⁴ mol/L), an analyte ClO₄⁻ (2.5×10⁻ mol/L), an analyte PO₄³⁻ (2.5×10⁻⁴ mol/L), an analyte H₂PO₄⁻ (2.5×10⁻⁴ mol/L), an analyte HPO₄²⁻ (2.5×10⁻⁴ mol/L), an analyte ppi (2.5×10⁻⁴ mol/L), an analyte SO₄²⁻ (2.5×10⁻⁴ mol/L), an analyte HSO₃⁻ (2.5×10⁻⁴ mol/L), an analyte S₂⁻ (2.5×10⁻⁴ mol/L), an analyte Ca²⁺ (2.5×10⁻⁴ mol/L), an analyte Mg²⁺ (2.5×10⁻⁴ mol/L), an analyte K⁺ (2.5×10⁻⁴ mol/L), an analyte L-Cys (2.5×10⁻⁴ mol/L), an analyte GSH (2.5×10⁻⁴ mol/L), an analyte ClO⁻ (2.5×10⁻⁴ mol/L), an analyte H₂O₂ (2.5×10⁻⁴ mol/L), an analyte ONOO⁻ (2.5×10⁻⁴ mol/L), an analyte ¹O₂ (2.5×10⁻⁴ mol/L), and an analyte ROO⁻ (2.5×10⁻⁴ mol/L). A fluorescence spectrometer with an excitation wavelength of 630 nm is used to analyze, and a fluorescence spectrogram is shown in FIG. 4A.

Solutions of EtOH:PBS (0.02 mol/L, pH=7.4, 5:95, v:v) of the rhodamine-methylene blue derivative fluorescent probe (1×10⁻⁵ mol/L) are respectively added with an analyte PO₄³⁻ (1×10⁻² mol/L), an analyte H₂PO₄⁻ (1×10⁻² mol/L), an analyte HPO₄²⁻ (1×10⁻² mol/L), an analyte ppi (1×10⁻² mol/L), an analyte L-Cys (1×10⁻² mol/L), an analyte GSH (1×10⁻² mol/L), an analyte ATP (1×10⁻² mol/L), an analyte ADP (1×10⁻² mol/L), an analyte AMP (1×10⁻² mol/L), an analyte GTP (1×10⁻² mol/L), an analyte CTP (1×10⁻² mol/L) and an analyte UDP (1×10⁻² mol/L). The fluorescence spectrometer with an excitation wavelength of 360 nm is used to analyze, and a fluorescence spectrogram is shown in FIG. 4B.

Referring to FIG. 4A and FIG. 4B, the rhodamine-methylene blue derivative fluorescent probe has obvious responses only to the ClO⁻ and the ATP, thus fluorescence signals can be used for rapid identification of the ClO⁻ and the ATP, while no change occurs in other analytes to be tested.

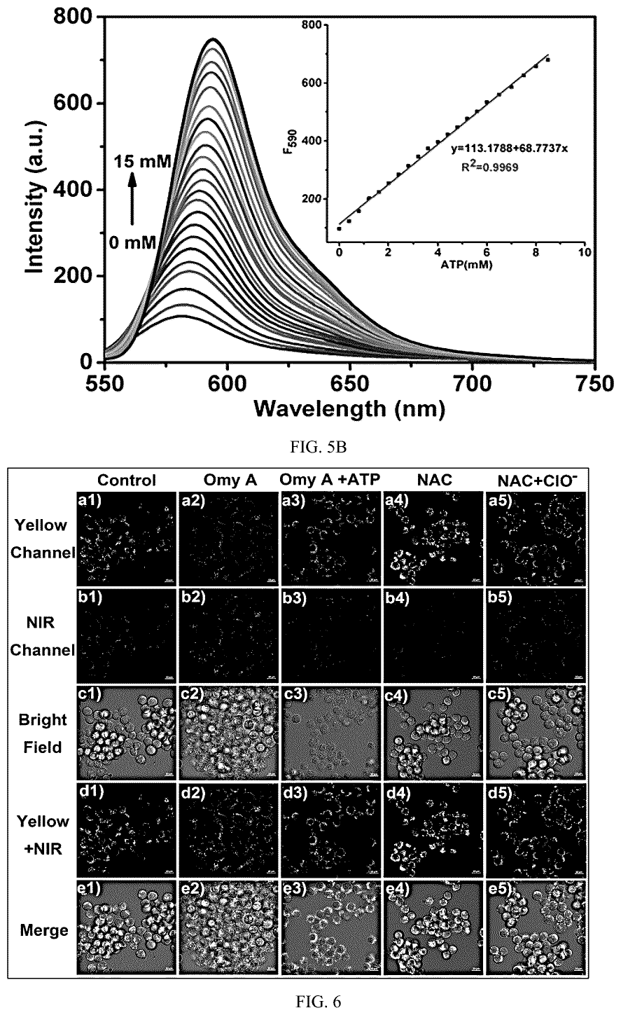

Solutions of EtOH:PBS (0.05 mol/L, pH=7.4, 5:95, v:v) of the rhodamine-methylene blue derivative fluorescent probe (1×10⁻⁵ mol/L) are gradually added with ClO⁻ and ATP solutions, respectively; and a concentration of the ClO⁻ is finally controlled to be 2.5×10⁻⁴ mol/L and a concentration of the ATP is finally controlled to be 1.5×10⁻² mol/L. Referring to FIG. 5A, fluorescence intensity of the rhodamine-methylene blue derivative fluorescent probe at 680 nm gradually increases with an increase of the concentration of the ClO⁻. Referring to FIG. 5B, the fluorescence intensity of the rhodamine-methylene blue derivative fluorescent probe at 590 nm gradually increases with an increase of the concentration of the ATP. Through calculation, it can be concluded that a detection limit of the rhodamine-methylene blue derivative fluorescent probe for the ClO⁻ is 0.90 nM, and for the ATP is 23.60 nM. Therefore, the rhodamine-methylene blue derivative fluorescent probe in the disclosure can be used for quantitative fluorescence detection of the ClO⁻ and the ATP.

### Embodiment 3

Detection experiments are below for ClO⁻ and ATP in cells by a fluorescent probe, that is, a rhodamine-methylene blue derivative fluorescent probe:

After Raw246.7 cells are incubated with relevant steps, fluorescence imaging is performed by an Olympus FV500-IX70 laser confocal microscope, and results of the fluorescence imaging are shown in FIG. 6. Control group (Control): the Raw246.7 cells are incubated with the fluorescent probe (1×10⁻⁵ mol/L) for 30 minutes (min). Oligomycin A (Omy A) group: the Raw246.7 cells are incubated with Omy A (2×10⁻⁵ mol/L) for 1 h and then incubated with the fluorescent probe (1×10⁻⁵ mol/L) for 30 min. Omy A+ATP group: the Raw246.7 cells are incubated with Omy A (2×10⁻⁵ mol/L) for 1 h to obtain incubated cells, the incubated cells are incubated with the ATP (1×10⁻³ mol/L) for 1 h and then incubated with the fluorescent probe (1×10⁻⁵ mol/L) for 30 min. N-acetylcysteine (NAC) group: the Raw246.7 cells are incubated with NAC (5×10⁻⁴ mol/L) for 1 h and then incubated with the fluorescent probe (1×10⁻⁵ mol/L) for 30 min. NAC+ClO⁻ group: the Raw246.7 cells are incubated with NAC (5×10⁻⁴ mol/L) for 1 h to obtain incubated cells, the incubated cells are incubated with ClO⁻ (2×10⁻⁴ mol/L) for 1 h and then incubated with the fluorescent probe (1×10⁻⁵ mol/L) for 30 min. After incubation of the Raw246.7 cells in the control group with the fluorescent probe, a yellow channel and a NIR channel show some fluorescent signals, indicating that a certain level of the ClO⁻ and the ATP is maintained in cells. When an ATP level is inhibited by Omy A, fluorescence of the yellow channel is significantly weakened and the fluorescence of the NIR channel is significantly enhanced. After using ATP to incubate the cells incubated with Omy A, the fluorescence of the yellow channel and the NIR channel is restored to a level of the control group, indicating that the fluorescent probe can detect exogenous ATP in cells. When a ClO⁻ level is inhibited by NAC, the fluorescence of the yellow channel is significantly enhanced, and the fluorescence of the NIR channel is significantly weakened. After using ClO⁻ to incubate the cells incubated with NAC, the fluorescence of the yellow channel and the NIR channel is restored to the level of the control group, indicating that the fluorescent probe can detect exogenous ClO⁻ in cells.

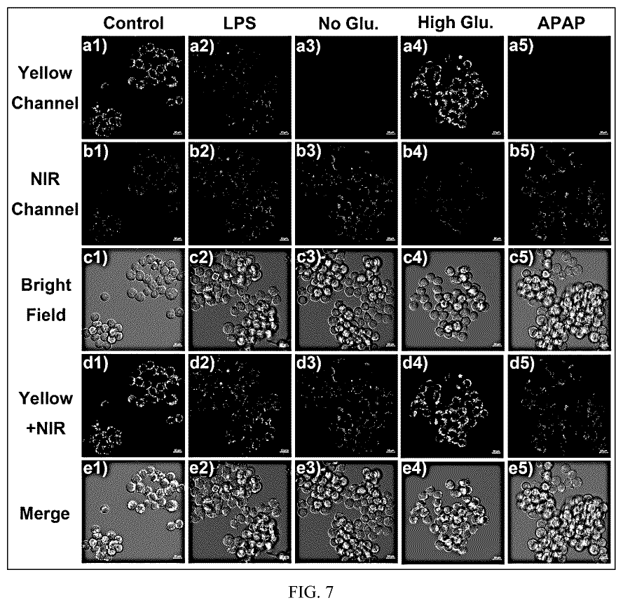

After the Raw246.7 cells are incubated with relevant steps, the fluorescence imaging is performed by the Olympus FV500-IX70 laser confocal microscope, and results of the fluorescence imaging are shown in FIG. 7. Control group (Control): the Raw246.7 cells are incubated with the fluorescent probe (1×10⁻⁵ mol/L) for 30 min, and a concentration of glucose (Glu) in a culture media is 5.5×10⁻³ mol/L. Lipopolysaccharide (LPS) group: the Raw246.7 cells are incubated with LPS (5 g/mL) for 1 h and then incubated with the fluorescent probe (1×10⁻⁵ mol/L) for 30 min. No Glu group: the Raw246.7 cells are incubated with the fluorescent probe (1×10⁻⁵ mol/L) for 30 min, and no Glu is added to the culture media. High Glu group: the Raw246.7 cells are incubated with the fluorescent probe (1×10⁻⁵ mol/L) for 30 min, and the concentration of Glu in the culture media is 2.5×10⁻² mol/L. Acetaminophen (C₈H₉NO₂, APAP) group: the Raw246.7 cells are incubated with 5×10⁻⁵ APAP for 1 h and then incubated with the fluorescent probe (1×10⁻⁵ mol/L) for 30 min. After incubation of cells in the control group with the fluorescent probe, there are some fluorescent signals in both the yellow channel and the NIR channel, indicating that the certain level of the ClO⁻ and the ATP is maintained in cells. When the LPS is used to stimulate cells to produce the ClO⁻, the fluorescence of the yellow channel is significantly weakened and the fluorescence of the NIR channel is significantly enhanced, indicating that the fluorescent probe can detect endogenous ClO⁻. When the Raw246.7 cells are incubated with the fluorescent probe in no Glu group, the yellow channel is almost non-fluorescent, and the fluorescence of the NIR channel is significantly enhanced; Conversely, when the Raw246.7 cells are incubated with the fluorescent probe in high Glu group, the fluorescence of the yellow channel is significantly enhanced, and the fluorescence of the NIR channel is significantly weakened, it is indicated that the fluorescent probe can detect endogenous ATP and that impaired ATP metabolism in the cells leads to an increase in a concentration of the ClO⁻. After stimulating the Raw246.7 cells with the APAP, the yellow channel is almost non-fluorescent and the NIR channel fluorescence is significantly enhanced, indicating that the APAP leads to an increase in the concentration of the ClO⁻ and an inhibition in ATP production, therefore, it is suggested that the fluorescent probe can be used for simultaneous fluorescence imaging of ClO⁻ and ATP in drug-stimulated cells.

The embodiments listed in the disclosure are only intended to illustrate the disclosure and are not intended to limit its scope. Any obvious amendments or modifications made by those skilled in the art to the disclosure shall not depart from the spirit and scope of the disclosure.

What is claimed is:

1. A rhodamine-methylene blue derivative fluorescent probe, having the following structural formula:

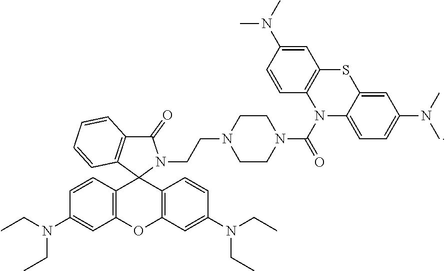 [SMILES: CCN(CC)c1ccc2c(c1)Oc1cc(N(CC)CC)ccc1C21c2ccccc2C(=O)N1CCN1CCN(C(=O)N2c3ccc(N(C)C)cc3Sc3cc(N(C)C)ccc32)CC1]

2. A preparation method of the rhodamine-methylene blue derivative fluorescent probe as claimed in claim 1, comprising:
dissolving methylene blue-carbonyl chloride (MB-COCl) and sodium carbonate in an organic solvent to obtain a first solution;
adding an organic solution including rhodamine B-(2-aminoethyl) piperazine (RhB-AP) to the first solution to obtain a second solution;
stirring the second solution for reaction to obtain a reacted solution;
adding the reacted solution to iced water and then extracting the reacted solution to obtain an extracted product; and
separating organic phases from the extracted product, followed by removing a solvent by rotary evaporation from the organic phases and then purifying the organic phases to obtain the rhodamine-methylene blue derivative fluorescent probe.

3. The preparation method of the rhodamine-methylene blue derivative fluorescent probe as claimed in claim 2, wherein a molar ratio of the MB-COCl to the sodium carbonate is 1:3.

4. The preparation method of the rhodamine-methylene blue derivative fluorescent probe as claimed in claim 2, wherein a weight-volume ratio of the MB-COCl to the organic solvent is 1 gram (g):10 milliliters (mL), and the organic solvent comprises dichloromethane.

5. The preparation method of the rhodamine-methylene blue derivative fluorescent probe as claimed in claim 2, wherein a molar ratio of the RhB-AP to the MB-COCl is 4:1, and a solvent for the organic solution including the RhB-AP comprises dichloromethane at a concentration of 2.296 mole per liter (mol·L⁻¹).

6. The preparation method of the rhodamine-methylene blue derivative fluorescent probe as claimed in claim 2, wherein conditions of the stirring are stirring the second solution for 5 hours at room temperature with a stirring frequency of 600 revolutions per minute (rpm).

7. The preparation method of the rhodamine-methylene blue derivative fluorescent probe as claimed in claim 2, wherein ethyl acetate is used for the extracting, conditions for the rotary evaporation comprise: a temperature of 50 degrees Celsius (° C.) and a pressure of 0.1 megapascal (MPa), and the purifying uses silica gel column chromatography.

8. A fluorescence quantitative detection method for hypochlorite (ClO⁻) and adenosine triphosphate (ATP), comprising: detecting the ClO⁻ and the ATP in cells by using the rhodamine-methylene blue derivative fluorescent probe as claimed in claim 1.

9. An application method of the rhodamine-methylene blue derivative fluorescent probe as claimed in claim 1, comprising: applying the rhodamine-methylene blue derivative fluorescent probe in detection of ClO⁻ and ATP.

10. An application method of the rhodamine-methylene blue derivative fluorescent probe as claimed in claim 1, comprising: applying the rhodamine-methylene blue derivative fluorescent probe as a ClO⁻ and ATP fluorescent probe in cell imaging.
